SEQUENCE LISTING (5 sequences)

SEQ ID NO: 1 - protein, 413 aa, Buttiauxella sp.
NDTPASGYQVEKVVILSRHGVRAPTKMTQTMRDVTPNTWPEWPVKLGYITPRGEHLISLMGGFYRQKFQQQGILSQGSCPTPNSIYVWADVDQRTLKTGEAFLAGLAPECHLTIHHQQDIKKADPLFHPVKAGTCSMDKTQVQQAVEKEAQTPIDNLNQHYIPFLALMNTTLNFSTSAWCQKHSADKSCDLGLSMPSKLSIKDNGNKVALDGAIGLSSTLAEIFLLEYAQGMPQAAWGNIHSEQEWASLLKLHNVQFDLMARTPYIARHNGTPLLQAISNALNPNATESKLPDISPDNKILFIAGHDTNIANIAGMLNMRWTLPGQPDNTPPGGALVFERLADKSGKQYVSVSMVYQTLEQLRSQTPLSLNQPAGSVQLKIPGCNDQTAEGYCPLSTFTRVVSQSVEPGCQLQ

SEQ ID NO: 2 - protein, 413 aa, synthetic construct
  REGION 1..413: note = phytase variant
NDTPASGYQVEKVVILSRHGVRAPTKMTQLMRDVTPNTWPEWPVPLGEITPRGEHLISLMGGFYRQKFQQLGILSQGSCPTPNSIYVWADVDQRTLKTGEAFLRGLAPECGLKIHHQQDIKKADPLFHPVKAGTCSMDKTQVQQAVEKEAQTPIDNLNQHYIPFLALMNTTLNFSTSAWCQKHSADKSCDLALSFPSKLSWKDNGNKVSLDGAIGLSSTLAEIFLLEYAQGMPQAAWGKIHSYQEWASLLKLHNVQFDLMARTPYIARHNGTPLLQAISNALNPNATESKLPDISPDNKILFIAGHDTNIANIAGMLNMRWTLPGQPDNTPPGGALNFERLADKSGKQYVSVSMVYQTLRQLRSAEPLSLNQPAGSVQLKIPGCNDQTAEGYCPLSTFTRVVSQSVEPGCQLQ

SEQ ID NO: 3 - protein, 386 aa, Talaromyces emersonii
  REGION 1..386: note = Xylanase
AGLNTAAKAIGLKYFGTATDNPELSDTAYETQLNNTQDFGQLTPANSMKWDATEPEQNVFTFSAGDQIANLAKANGQMLRCHNLVWYNQLPSWVTSGSWTNETLLAAMKNHITNVVTHYKGQCYAWDVVNEALNDDGTYRSNVFYQYIGEAYIPIAFATAAAADPNAKLYYNDYNIEYPGAKATAAQNLVKLVQSYGARIDGVGLQSHFIVGETPSTSSQQQNMAAFTALGVEVAITELDIRMQLPETEALLTQQATDYQSTVQACANTKGCVGITVWDWTDKYSWVPSTFSGYGDACPWDANYQKKPAYEGILTGLGQTVTSTTYIISPTTSVGTGTTTSSGGSGGTTGVAQHWEQCGGLGWTGPTVCASGYTCTVINEYYSQCL

SEQ ID NO: 4 - protein, 410 aa, synthetic construct
  REGION 1..410: note = Mannanase variant
ASRFVTISGTQFNIDGKVGYFAGTNCYWCSYLTNHADVDSTFSHISSSGLKVVRVWGFNDVNTQPPPGQIWFQKLSATGSTINTGADGLQTLDYVVRSAEQHNLKLIIPFVNYWSDYGGINAYVNAFGGNATTWYTNTAAQTQYRKYVQAVVSRYANSTAIFAWELGNEPRCHGCSTDVIHQWATSVSQYVKSLDSNHLVSLGDEGFGLSTGDGTYPYTYGEGTDFAKNVQIKSLDFGTFHLYPDSWGTNYTWGNGWIRTHAAACLAAGKPCVLEEYGARQDPCTNEAPWQTTSLTTRGMGGDMFWQWGDTFANGAQSNSDPYTVWYNSSSWQCLVKNHVDAINGGTTTPPPVSSTTTTSSRTSSTPPPPGGSCSPLYGQCGGSGYTGPTCCAQGTCIYSNYWYSQCLNT

SEQ ID NO: 5 - protein, 335 aa, Talaromyces emersonii
  REGION 1..335: note = Glucanase
MKFSRVVCGLTAAGGALAAPVKEKGIKKRASPFQWFGSNESGAEFGNNNIPGVEGTDYTFPNTSAIQILIDQGMNIFRVPFLMERMVPNQMTGPVDSAYFQGYSQVINYITSHGASAVIDPHNFGRYYNNIISSPSDFQTFWHTIASNFADNDNVIFDTNNEYHDMDESLVVQLNQAAIDGIRAAGATSQYIFVEGNSWTGAWTWTQVNDAMANLTDPQNKIVYEMHQYLDSDGSGTSDQCVNSTIGQDRVESATAWLKQNGKKAILGEYAGGANSVCETAVTGMLDYLANNTDVWTGAIWWAAGPWWGDYIFSMEPPSGIAYEQVLPLLKPYLE

(19) United States
(12) Patent Application Publication
(10) Pub. No.: US 2026/0209727 A1
(43) Pub. Date: Jul. 23, 2026

(54) PHYTASE VARIANTS WITH IMPROVED PROPERTIES

(71) Applicant: BASF SE, Ludwigshafen am Rhein (DE)

(72) Inventors: Arne Solbak, San Diego, CA (US); Stefan Haefner, Ludwigshafen am Rhein (DE)

(21) Appl. No.: 19/141,750

(22) PCT Filed: Dec. 21, 2023
(86) PCT No.: PCT/IB2023/063096

§ 371 Date: Jun. 20, 2025

(30) Foreign Application Priority Data
Dec. 23, 2022 (EP) 22216268.7

(51) Int. Cl. C12N 9/16; A23K 20/189

(52) U.S. Cl. CPC C12N 9/16; A23K 20/189

(57) ABSTRACT

The present invention relates to phytase variants with improved properties such as an increased thermostability and/or an increased gastric stability, nucleotide sequences encoding said phytases, processes for the preparation and the use of phytases and to animal feeds comprising these phytases.

### FIELD OF THE INVENTION

The present invention relates to phytase variants with improved properties such as an increased thermostability and/or an increased gastric stability, nucleotide sequences encoding said phytases, processes for the preparation and the use of phytases and to animal feeds comprising these phytases.

### BACKGROUND OF THE INVENTION

Phosphorus is an essential element for the growth of living organisms. In animal production, feed, as a rule, has to be supplemented with inorganic phosphorus in order to achieve good growth rates. In cereals and pulses, phosphorus is stored mainly in the form of phytate. However, monogastric animals such as pigs, poultry and fish are not capable of directly absorbing phytate or phytic acid, which results in the excretion of phytate, which means phosphorus overloads in regions with intensive livestock production. Furthermore, phytic acid, which binds metals such as calcium, copper or zinc, acts as a substance with a negative effect on the metabolism of monogastric animals. In order to compensate for the phosphate deficit of these animals and to ensure sufficient growth and sufficient health, inorganic phosphate is added to the animal feed.

This addition of inorganic phosphate is costly and leads to a further adverse effect on the environment. By using a phytase in animal feeds, the phytate is hydrolyzed and results in a lower content of inositol phosphate and inorganic phosphates in the slurry. The addition of phytases to animal feeds improves the availability of organic phosphorus and reduces the adverse effects on the environment by excreted, phytate-bound phosphates.

Phytases, also referred to as myo-inositol hexakisphosphate phosphohydrolases, are a class of phosphatases which are capable of cleaving at least one phosphate residue from phytate.

EP 420 358 generally describes the cloning and expression of microbial phytases, WO 2006/38062 describes microbial phytases derived from Citrobacter freundii as additive to animal feeds, and WO 2007/112739 describes phytases based on a natural phytase from Citrobacter braakii and processes for its preparation and the use in animal feeds.

Haefner et aL. (2005) Appi Microbiol Biotechnol 68:588-597 describe a multiplicity of known uses of phytases in the field of human or animal nutrition. Further uses of phytases such as, for example, the use for hydrolyzing biomass or starch in the production of bioethanol are described in WO 2008/097620.

WO 2008/116878 and WO 2010/034835 describe a phytase from Hafnia a/vei, its protein sequence and variants thereof. Zinin et al. (2004) FEMS Microbiology Letters 236:283-290) disclose a phytase from Obesumbacterium proteus, whose sequence is deposited at the UNIPROT database with the accession number Q6U677. The patent applications WO 2006/043178, WO 2008/097619 and WO 2008/092901 describe phytases from various Buttiauxella sp. The natural phytases with the currently highest specific activities include the natural phytases from Yersinia intermedia (WO 2007/128160) and Yersinia pestis (WO 02/048332).

WO 2011/048046 discloses a synthetic phytase obtained by ligating parts of the DNA encoding a phytase from Hafnia sp. LU11047 and of the DNA encoding a putative phytase from Yersinia mollaretii. WO 2012/143861 and WO 2012/143862 disclose variants of this synthetic phytase with increased thermal stability which are stable over a wide pH range.

WO 2020/106796 and WO 2021/102238 disclose engineered phytase polypeptides.

However, all of these currently available phytases do not show those properties which are required for the preparation of animal feed additives. The currently available phytases are not sufficiently thermally stable for being employed in the preparation of animal feed pellets without a considerable loss of their activity. In the preparation of animal feed pellets, phytase together with further customary animal feed components is compressed under high temperatures and humidity in order to be fed to the livestock as one entity. An effective destruction of Salmonella sp. and the gelatinization of the starch is only achieved above a temperature of 80° C. during the preparation (Amerah et al. Worlds Poulty Science Journal (2011) 67:29-45). This compressing under hot and humid conditions results in considerable phytase activity losses. One possibility of preventing this loss of activity is the laborious coating of the phytase particles, so that they are protected against the effect of heat. This coating of the phytase additions causes considerable additional costs as the result of the fats or polymers employed for the coating.

Further, the doses of commercial phytases are usually determined on the basis of the activity determination at pH 5.5 (DIN ISO 30024:2009) and are not adapted to match the pH in the respective digestive tract. This results in considerable misdosages by variation of the activity at pH values other than 5.5.

Finally, for a complete degradation of phytic acid and the release of phosphate it is desirable to use a phytase enzyme which does not only efficiently act on phytic acid, but also on the lower phytate esters (inositol x-phosphate or Ipx, with x being <6).

Hence, there is still a need for improved phytases which show an enhanced thermostability, a better stability at low pH and an enhanced activity against lower phytate esters.

### SUMMARY OF THE INVENTION

The present invention provides phytase variants with increased thermostability, better stability at low pH and an enhanced activity against lower phytate esters compared to the prior art phytases.

Hence, in one aspect the present invention relates to a polypeptide having phytase activity or fragment thereof having phytase activity, wherein the polypeptide or fragment thereof has an amino acid sequence which is at least 94% identical to the amino acid sequence as set forth in SEQ ID NO: 1 and wherein the polypeptide comprises at least one amino acid modification at an amino acid residue number selected from the group consisting of 71, 104, 192, 195, 201, 239, 243, 337, 360 and 365, compared to the amino acid sequence as set forth in SEQ ID NO: 1 and referring to the numbering of SEQ ID NO: 1.

In one embodiment, the at least one amino acid modification is an amino acid substitution selected from the group consisting of 71L, 104R, 192A, 195F, 201W, 239K, 243Y, 337N, 360R and 365A.

In one embodiment, the polypeptide having phytase activity or fragment thereof having phytase activity has at least one additional amino acid modification at an amino acid residue number selected from the group consisting of 30, 45, 48, 111, 113, 209 and 366 compared to the amino acid sequence as set forth in SEQ ID NO: 1 and referring to the numbering of SEQ ID NO: 1, preferably wherein the at least one additional amino acid modification is an amino acid substitution selected from the group consisting of 30L, 45P, 48E, 111 G, 113K, 209S and T366E.

In one embodiment, the polypeptide having phytase activity or fragment thereof having phytase activity has at least one additional amino acid modification at an amino acid residue number selected from the group consisting of 2, 76, 78, 89, 121, 138, 143, 152, 164, 200, 202, 207, 234, 242, 244, 247, 251, 256, 258, 261, 270, 314, 355 and 372 compared to the amino acid sequence as set forth in SEQ ID NO: 1 and referring to the numbering of SEQ ID NO: 1, preferably wherein the at least one additional amino acid modification is an amino acid substitution selected from the group consisting of 2E, 76N, 78T, 89T, 121T, 138N, 143Y, 152G, 164E, 200N, 202S, 207E, 234V, 242N, 244S, 247K, 251N, 256H, 258N, 261H, 270Q, 314G, 355I and 372N.

The present invention also relates to a polypeptide having phytase activity or fragment thereof having phytase activity, wherein the polypeptide or fragment thereof has a melting temperature Tm of at least 101.5° C. as determined by differential scanning calorimetry using the conditions as described in Example 1.2.

The present invention also relates to a polypeptide having phytase activity or fragment thereof having phytase activity, wherein the polypeptide or fragment thereof has an amino acid sequence which is at least 95.9% identical to the amino acid sequence as set forth in SEQ ID NO: 2.

The present invention also relates to an isolated nucleic acid molecule encoding said polypeptide or fragment thereof having phytase activity, an expression vector comprising said nucleic acid molecule and a host cell comprising said nucleic acid molecule or said expression vector.

The present invention also relates to a method for producing said polypeptide or fragment thereof having phytase activity, comprising culturing said host cell under suitable conditions, optionally further comprising recovering the polypeptide or fragment thereof.

The present invention also relates to an animal feed additive or animal feed comprising said polypeptide or fragment thereof having phytase activity, optionally further comprising at least one additional enzyme, optionally wherein the at least one additional enzyme is selected from the group consisting of cellulase, xylanase, glucanase, mannanase, protease, amylase and a second phytase.

The present invention also relates to the use of said polypeptide or fragment thereof having phytase activity or of said animal feed additive in an animal feed or for reducing the phosphate content in the slurry of livestock or for improving animal performance.

The present invention also relates to the use of said animal feed for reducing the phosphate content in the slurry of livestock or for improving animal performance.

### DETAILED DESCRIPTION OF THE INVENTION

Although the present invention will be described with respect to particular embodiments, this description is not to be construed in a limiting sense.

Before describing in detail exemplary embodiments of the present invention, definitions important for understanding the present invention are given. Unless stated otherwise or apparent from the nature of the definition, the definitions apply to all methods and uses described herein.

As used in this specification and in the appended claims, the singular forms of “a” and “an” also include the respective plurals unless the context clearly dictates otherwise. In the context of the present invention, the terms “about” and “approximately” denote an interval of accuracy that a person skilled in the art will understand to still ensure the technical effect of the feature in question. The term typically indicates a deviation from the indicated numerical value of ±20%, preferably ±15%, more preferably ±10%, and even more preferably ±5%.

It is to be understood that the term “comprising” is not limiting. For the purposes of the present invention the term “consisting of” is considered to be a preferred embodiment of the term “comprising”. If hereinafter a group is defined to comprise at least a certain number of embodiments, this is meant to also encompass a group which preferably consists of these embodiments only.

Furthermore, the terms “first”, “second”, “third” or “(a)”, “(b)”, “(c)”, “(d)” etc. and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein. In case the terms “first”, “second”, “third” or “(a)”, “(b)”, “(c)”, “(d)”, “i”, “ii” etc. relate to steps of a method or use or assay there is no time or time interval coherence between the steps, i.e. the steps may be carried out simultaneously or there may be time intervals of seconds, minutes, hours, days, weeks, months or even years between such steps, unless otherwise indicated in the application as set forth herein above or below.

It is to be understood that this invention is not limited to the particular methodology, protocols, reagents etc. described herein as these may vary. It is also to be understood that the terminology used herein is for the purpose of describing particular embodiments only and is not intended to limit the scope of the present invention that will be limited only by the appended claims. Unless defined otherwise, all technical and scientific terms used herein have the same meanings as commonly understood by one of ordinary skill in the art.

As discussed above, the present invention provides polypeptides having phytase activity with an increased thermostability and an increased stability at low pH. The polypeptides of the present invention also have a high activity against lower phytate esters.

The terms “peptides”, “proteins” and “polypeptides” are used interchangeably herein and refer to a polymer of amino acids joined together by peptide bonds. A “protein” or “polypeptide” comprises a polymeric sequence of amino acid residues. The single and 3-letter code for amino acids as defined in conformity with the IUPAC-IUB Joint Commission on Biochemical Nomenclature (JCBN) is used throughout this disclosure. The single letter X refers to any of the twenty amino acids. It is also understood that a polypeptide may be coded for by more than one nucleotide sequence due to the degeneracy of the genetic code. Within the present disclosure, a polypeptide having one or more amino acid modifications compared to another polypeptide may also be designated as “variant polypeptide”.

A “phytase”, also called myo-inositol hexakisphosphate phosphohydrolase, is an enzyme that catalyzes the hydrolysis of phytate (myo-inositol hexakisphosphate or IP6), thereby releasing a usable form of inorganic phosphorus. In one embodiment the phytase is able to catalyze the release of one phosphate molecule from myo-inositol hexakisphosphate (so-called IP6 activity).

The phytase of the present invention is also able to catalyze the release of one phosphate molecule from inositol phosphate with five phosphates (so-called IP5 activity), and/or is able to catalyze the release of one phosphate molecule from inositol phosphate with four phosphates (so-called IP4 activity) and/or is able to catalyze the release of one phosphate molecule from inositol phosphate with three phosphates (so-called IP3 activity).

The phytase activity can be determined by incubating a polypeptide or a phytase with a substrate such as sodium phytate under suitable conditions, such as 100 mM Na acetate, pH 4.5, for a suitable period. One example for determining phytase activity is described in the example section herein. One unit of phytase activity (FTU) is defined as the amount of enzyme required to release 1 mole of inorganic phosphate under the conditions of the assay. A standardized assay for determining phytase activity can be taken from ISO 30024:2009. Further, an overview of methods for determining phytase activity is provided in Qvirist et al. (2015) J. Biol. Methods 2(1): e16(1).

A “fragment” of the polypeptide having phytase activity refers to a portion or subsequence of the phytase polypeptide which retains essentially the same phytase activity as the polypeptide having phytase activity. The fragment of the polypeptide having phytase activity may have a length of 200 to 413 amino acids or 210 to 413 amino acids or 220 to 413 amino acids or 230 to 413 amino acids or 240 to 413 amino acids or 250 to 413 amino acids or 260 to 413 amino acids or 270 to 413 amino acids or 280 to 413 amino acids or 290 to 413 amino acids, preferably it has a length of 300 to 413 amino acids or 310 to 413 amino acids or 320 to 413 amino acids or 330 to 413 amino acids or 340 to 413 amino acids or 350 to 413 amino acids, more preferably it has a length of 355 to 413 amino acids, 360 to 413 amino acids, 365 to 413 amino acids, 370 to 413 amino acids, 375 to 413 amino acids, 380 to 413 amino acids or 385 to 413 amino acids. Most preferably, the fragment of the polypeptide having phytase activity has a length of 390 to 413 amino acids, 395 to 413 amino acids, 397 to 413 amino acids, 400 to 413 amino acids, 402 to 413 amino acids or 405 to 413 amino acids.

“Essentially the same phytase activity” means that the fragment has at least 80%, 81%, 82%, 83%, 84%, 85%, 86%, 87%, 88%, 89% or 90%, preferably at least 91%, 92%, 93%, 94% or 95% and most preferably at least 96%, 97%, 98% or 99% of the phytase activity of the phytase according to SEQ ID NO: 1. The phytase activity is determined as described in Example 1.1 herein.

“Sequence Identity”, “% sequence identity”, “% identity”, “% identical” or “sequence alignment” means a comparison of a first amino acid sequence to a second amino acid sequence, or a comparison of a first nucleic acid sequence to a second nucleic acid sequence and is calculated as a percentage based on the comparison. The result of this calculation can be described as “percent identical” or “percent ID.”

Generally, a sequence alignment can be used to calculate the sequence identity by one of two different approaches. In the first approach, both mismatches at a single position and gaps at a single position are counted as non-identical positions in final sequence identity calculation. In the second approach, mismatches at a single position are counted as non-identical positions in final sequence identity calculation; however, gaps at a single position are not counted (ignored) as non-identical positions in final sequence identity calculation. In other words, in the second approach gaps are ignored in final sequence identity calculation. The difference between these two approaches, i.e. counting gaps as non-identical positions vs ignoring gaps, at a single position can lead to variability in the sequence identity value between two sequences.

A sequence identity can be calculated from a pairwise alignment showing both sequences over the full length, so showing the first sequence and the second sequence in their full length (“Global sequence identity”). For example, program Needle (EMBOSS) produces such alignments; % sequence identity=(# of identical residues/length of alignment)×100)].

A sequence identity can be calculated from a pairwise alignment showing only a local region of the first sequence or the second sequence (“Local Identity”). For example, program Blast (NCBI) produces such alignments; % sequence identity=(# of Identical residues/length of alignment)×100)].

According to the present invention, sequence identity is calculated wherein mismatches at a single position are counted as non-identical positions in final sequence identity calculation; however, gaps at a single position are not counted (ignored) as non-identical positions in final sequence identity calculation. The sequence alignment is generated by using the algorithm of Needleman and Wunsch (J. Mol. Biol. (1979) 48, p. 443-453). Preferably, the program “NEEDLE” (The European Molecular Biology Open Software Suite (EMBOSS)) is used with the programs default parameter (gap open=10.0, gap extend=0.5 and matrix=EBLOSUM62). Then, a sequence identity can be calculated from the alignment showing both sequences over the full length, so showing the first sequence and the second sequence in their full length (“Global sequence identity”) and the output of Needle labeled “longest identity” is used as percent identity. For example: % sequence identity=(# of identical residues/length of alignment minus gaps)×100)].

The term “amino acid modification” means that the amino acid sequence of the variant polypeptide is modified compared to the amino acid sequence of the parent polypeptide, i.e. the polypeptide according to SEQ ID No: 1. The term “amino acid modification” is not intended to comprise modifications to an amino acid residue itself, such as, but not limited to, phosphorylation, myristoylation, palmitoylation, isoprenylation, acetylation, alkylation, amidation, gamma-carboxylation or glycosylation. The term “amino acid modification” includes an amino acid substitution, amino acid insertion and amino acid deletion. Hence, the variant polypeptide of the present invention comprises at least one amino acid substitution, amino acid insertion and/or amino acid deletion compared to the parent polypeptide, i.e. the polypeptide according to SEQ ID No: 1. Preferably, the amino acid modification is an amino acid substitution.

An “amino acid substitution” may be described by providing the original amino acid residue in the parent polypeptide followed by the number of the position of this amino acid residue within the amino acid sequence. For example, a substitution of amino acid residue 71 means that the amino acid of the parent at position 71 can be substituted with any of the 19 other amino acid residues and is designated as “71”. In addition, a substitution can be described by providing the original amino acid residue in the parent polypeptide. For example, the substitution of serine at residue 71 is designated as “Gln71” or “Q71”. In addition, a substitution can be described by providing the original amino acid residue in the parent polypeptide followed by the number of the position of this amino acid residue within the amino acid sequence and followed by the specific substituted amino acid within the variant polypeptide. For example, the substitution of glutamine at position 71 with leucine is designated as “Gln71 Leu” or “Q71 L”. In addition, a substitution can be described by providing the number of the position of this amino acid residue within the amino acid sequence and followed by the specific substituted amino acid within the variant polypeptide. For example, the substitution at position 71 with leucine is designated as “71 Leu” or “71 L”. If more than one specific amino acid substitution follows the position number, e.g. “71 LN”, the parent amino acid at the indicated position (here: position 71) can be substituted by any one of the listed substituted amino acids (here: either leucine or valine). Combinations of substitutions are described by inserting commas between the amino acid residues, for example: 71 L, 104R, 111G, 142V represents a combination of substitutions of four different amino acid residues when compared to a parent polypeptide. Variants having a substitution on the amino acid level are encoded by a nucleic acid sequence which differs from the parent nucleic acid sequence encoding the parent polypeptide at least in the position encoding the substituted amino acid residue.

The amino acid substitution in the variant polypeptide may be a conservative amino acid substitution. A “conservative amino acid substitution” or “substitution with a related amino acid” means replacement of one amino acid residue in an amino acid sequence with a different amino acid residue having a similar property at the same position compared to the parent amino acid sequence. Some examples of a conservative amino acid substitution include, but are not limited to, replacing a positively charged amino acid residue with a different positively charged amino acid residue; replacing a polar amino acid residue with a different polar amino acid residue; replacing a non-polar amino acid residue with a different non-polar amino acid residue, replacing a basic amino acid residue with a different basic amino acid residue, or replacing an aromatic amino acid residue with a different aromatic amino acid residue.

A list of conservative amino acid substitutions is provided in the Table below (see for example Creighton (1984) Proteins. W. H. Freeman and Company (Eds)).

| | Conservative |
|---|---|
| Residue | Substitution(s) |
| Ala | Ser |
| Arg | Lys |
| Asn | Gln, His |
| Asp | Glu |
| Gln | Asn |
| Cys | Ser |
| Glu | Asp |
| Gly | Pro |
| His | Asn, Gln |
| Ile | Leu, Val |
| Leu | Ile, Val |
| Lys | Arg, Gln |
| Met | Leu, Ile |
| Phe | Met, Leu, Tyr |
| Ser | Thr, Gly |
| Thr | Ser, Val |
| Trp | Tyr |
| Tyr | Trp, Phe |
| Val | Ile, Leu |

An “amino acid insertion” is described by providing the number of the position within the amino acid sequence behind which the amino acid is inserted followed by an apostrophe and the specific inserted amino acid residue. For example, the insertion of serine behind position 132 is designated as “132'S”. Variants having an insertion on the amino acid level are encoded by a nucleic acid sequence which differs from the parent nucleic acid sequence encoding the parent polypeptide at least in the position encoding the inserted amino acid residue.

An “amino acid deletion” is described by providing the number of the position within the amino acid sequence at which the amino acid residue is deleted followed by a delta and the specific deleted amino acid residue. For example, a deletion of asparagine on position 125 is designated as “125ΔN”. Variants having deletions on the amino acid level are encoded by a nucleic acid sequence which differs from the parent nucleic acid sequence encoding the parent polypeptide at least at the position encoding the deleted amino acid residue.

In one embodiment, the polypeptide having phytase activity or fragment thereof having phytase activity has an amino acid sequence which is at least 94% identical to the amino acid sequence as set forth in SEQ ID NO: 1 and wherein the polypeptide comprises at least one amino acid modification at an amino acid residue number selected from the group consisting of 71, 104, 192, 195, 201, 239, 243, 337, 360 and 365, compared to the amino acid sequence as set forth in SEQ ID NO: 1 and referring to the numbering of SEQ ID NO: 1. % identity is determined using the Needle program of the EMBOSS package (version 6.3.1.2 or later) with a gap open penalty of 10, a gap extension penalty of 0.5, and the EBLOSUM62 by the calculation: (Identical Residues×100)/(Length of Alignment−Total Number of Gaps in Alignment), which is also called “Longest_Identity” in programs output when parameter “-nobrief” is applied.

The polypeptide having phytase activity is at least 94.5% identical, at least 95% identical, at least 95.5% identical, at least 96% identical, at least 96.5% identical, at least 97% identical, at least 97.5% identical, at least 98% identical, at least 98.5% identical or at least 99% identical to the full length amino acid sequence of the phytase according to SEQ ID NO: 1.

The polypeptide having phytase activity has less than 80, 78, 76, 74, 72, 70, 68, 66, 64, 62 or 60 amino acid modifications, less than 58, 56, 54, 52, 50, 48, 46, 44, 42 or 40 amino acid modifications, less than 38, 36, 34, 32, 30, less than 29, 28, 27, 26 or 25 amino acid modifications compared to the full length amino acid sequence of the phytase according to SEQ ID NO: 1.

The polypeptide having phytase activity has less than 80, 78, 76, 74, 72, 70, 68, 66, 64, 62 or 60 amino acid substitutions, less than 58, 56, 54, 52, 50, 48, 46, 44, 42 or 40 amino acid substitutions, less than 38, 36, 34, 32, 30, less than 29, 28, 27, 26 or 25 amino acid substitutions compared to the full length amino acid sequence of the phytase according to SEQ ID NO: 1.

The polypeptide having phytase activity is further characterized in that it comprises at least one amino acid modification at an amino acid residue number selected from the group consisting of 71, 104, 192, 195, 201, 239, 243, 337, 360 and 365, compared to the amino acid sequence as set forth in SEQ ID NO: 1 and referring to the numbering of SEQ ID NO: 1.

The amino acid positions which are modified can be identified by aligning the amino acid sequence according to SEQ ID NO: 1 with the amino acid sequence containing the amino acid modification(s) using the algorithms described above.

Preferably, the polypeptide having phytase activity comprises at least two, at least three or at least four amino acid modifications at an amino acid residue number selected from the group consisting of 71, 104, 192, 195, 201, 239, 243, 337, 360 and 365, compared to the amino acid sequence as set forth in SEQ ID NO: 1 and referring to the numbering of SEQ ID NO: 1. More preferably, the polypeptide having phytase activity comprises or at least five, at least six or at least seven amino acid modifications at an amino acid residue number selected from the group consisting of 71, 104, 192, 195, 201, 239, 243, 337, 360 and 365, compared to the amino acid sequence as set forth in SEQ ID NO: 1 and referring to the numbering of SEQ ID NO: 1. Even more preferably, the polypeptide having phytase activity comprises at least eight or at least nine amino acid modifications at an amino acid residue number selected from the group consisting of 71, 104, 192, 195, 201, 239, 243, 337, 360 and 365, compared to the amino acid sequence as set forth in SEQ ID NO: 1 and referring to the numbering of SEQ ID NO: 1. Most preferably, the polypeptide having phytase activity comprises an amino acid modification at all amino residue numbers 71, 104, 192, 195, 201, 239, 243, 337, 360 and 365, compared to the amino acid sequence as set forth in SEQ ID NO: 1 and referring to the numbering of SEQ ID NO: 1.

In one embodiment, the polypeptide having phytase activity comprises at least one amino acid modification at an amino acid residue number selected from the group consisting of 192, 195, 360 and 365, compared to the amino acid sequence as set forth in SEQ ID NO: 1 and referring to the numbering of SEQ ID NO: 1.

In one embodiment, the polypeptide having phytase activity comprises an amino acid modification at amino acid residue number 195 and optionally one or more additional amino acid modifications at an amino acid residue number selected from the group consisting of 71, 104, 192, 201, 239, 243, 337, 360 and 365, compared to the amino acid sequence as set forth in SEQ ID NO: 1 and referring to the numbering of SEQ ID NO: 1.

In one embodiment, the polypeptide having phytase activity comprises an amino acid modification at amino acid residue number 365 and optionally one or more additional amino acid modifications at an amino acid residue number selected from the group consisting of 71, 104, 192, 195, 201, 239, 243, 337 and 360, compared to the amino acid sequence as set forth in SEQ ID NO: 1 and referring to the numbering of SEQ ID NO: 1.

In one embodiment, the polypeptide having phytase activity comprises an amino acid modification at amino acid residue number 360 and optionally one or more additional amino acid modifications at an amino acid residue number selected from the group consisting of 71, 104, 192, 195, 201, 239, 243, 337 and 365, compared to the amino acid sequence as set forth in SEQ ID NO: 1 and referring to the numbering of SEQ ID NO: 1.

In one embodiment, the polypeptide having phytase activity comprises an amino acid modification at amino acid residue number 243 and optionally one or more additional amino acid modifications at an amino acid residue number selected from the group consisting of 71, 104, 192, 195, 201, 239, 337, 360 and 365, compared to the amino acid sequence as set forth in SEQ ID NO: 1 and referring to the numbering of SEQ ID NO: 1.

In one embodiment, the polypeptide having phytase activity comprises an amino acid modification at amino acid residue number 192 and optionally one or more additional amino acid modifications at an amino acid residue number selected from the group consisting of 71, 104, 195, 201, 239, 243, 337, 360 and 365, compared to the amino acid sequence as set forth in SEQ ID NO: 1 and referring to the numbering of SEQ ID NO: 1.

The polypeptide having phytase activity is further characterized in that it comprises at least one amino acid substitution at an amino acid residue number selected from the group consisting of 71, 104, 192, 195, 201, 239, 243, 337, 360 and 365, compared to the amino acid sequence as set forth in SEQ ID NO: 1 and referring to the numbering of SEQ ID NO: 1.

Preferably, the polypeptide having phytase activity comprises at least two, at least three or at least four at an amino acid residue number selected from the group consisting of 71, 104, 192, 195, 201, 239, 243, 337, 360 and 365, compared to the amino acid sequence as set forth in SEQ ID NO: 1 and referring to the numbering of SEQ ID NO: 1. More preferably, the polypeptide having phytase activity comprises at least five, at least six or at least seven amino acid substitutions at an amino acid residue number selected from the group consisting of 71, 104, 192, 195, 201, 239, 243, 337, 360 and 365, compared to the amino acid sequence as set forth in SEQ ID NO: 1 and referring to the numbering of SEQ ID NO: 1. Even more preferably, the polypeptide having phytase activity comprises at least eight or at least nine amino acid substitutions at an amino acid residue number selected from the group consisting of 71, 104, 192, 195, 201, 239, 243, 337, 360 and 365, compared to the amino acid sequence as set forth in SEQ ID NO: 1 and referring to the numbering of SEQ ID NO: 1. Most preferably, the polypeptide having phytase activity comprises an amino acid substitution at all amino residue numbers 71, 104, 192, 195, 201, 239, 243, 337, 360 and 365, compared to the amino acid sequence as set forth in SEQ ID NO: 1 and referring to the numbering of SEQ ID NO: 1.

In one embodiment, the polypeptide having phytase activity comprises at least one amino acid substitution at an amino acid residue number selected from the group consisting of 192, 195, 243, 360 and 365.

In one embodiment, the polypeptide having phytase activity comprises an amino acid substitution at amino acid residue number 195 and optionally one or more additional amino acid substitutions at an amino acid residue number selected from the group consisting of 71, 104, 192, 201, 239, 243, 337, 360 and 365, compared to the amino acid sequence as set forth in SEQ ID NO: 1 and referring to the numbering of SEQ ID NO: 1.

In one embodiment, the polypeptide having phytase activity comprises an amino acid substitution at amino acid residue number 365 and optionally one or more additional amino acid substitutions at an amino acid residue number selected from the group consisting of 71, 104, 192, 195, 201, 239, 243, 337 and 360, compared to the amino acid sequence as set forth in SEQ ID NO: 1 and referring to the numbering of SEQ ID NO: 1.

In one embodiment, the polypeptide having phytase activity comprises an amino acid substitution at amino acid residue number 360 and optionally one or more additional amino acid substitutions at an amino acid residue number selected from the group consisting of 71, 104, 192, 195, 201, 239, 243, 337 and 365, compared to the amino acid sequence as set forth in SEQ ID NO: 1 and referring to the numbering of SEQ ID NO: 1.

In one embodiment, the polypeptide having phytase activity comprises an amino acid substitution at amino acid residue number 243 and optionally one or more additional amino acid substitutions at an amino acid residue number selected from the group consisting of 71, 104, 192, 195, 201, 239, 337, 360 and 365, compared to the amino acid sequence as set forth in SEQ ID NO: 1 and referring to the numbering of SEQ ID NO: 1.

In one embodiment, the polypeptide having phytase activity comprises an amino acid substitution at amino acid residue number 192 and optionally one or more additional amino acid substitutions at an amino acid residue number selected from the group consisting of 71, 104, 195, 201, 239, 243, 337, 360 and 365, compared to the amino acid sequence as set forth in SEQ ID NO: 1 and referring to the numbering of SEQ ID NO: 1.

In one embodiment, the polypeptide having phytase activity comprises at least one amino acid substitution selected from the group consisting of 71L, 104R, 192A, 195F, 201W, 239K, 243Y, 337N, 360R and 365A, compared to the amino acid sequence as set forth in SEQ ID NO: 1 and referring to the numbering of SEQ ID NO: 1.

Preferably, the polypeptide having phytase activity comprises at least two, at least three or at least four amino acid substitutions selected from the group consisting of 71 L, 104R, 192A, 195F, 201W, 239K, 243Y, 337N, 360R and 365A, compared to the amino acid sequence as set forth in SEQ ID NO: 1 and referring to the numbering of SEQ ID NO: 1. More preferably, the polypeptide having phytase activity comprises at least five, at least six or at least seven amino acid substitutions selected from the group consisting of 71L, 104R, 192A, 195F, 201W, 239K, 243Y, 337N, 360R and 365A, compared to the amino acid sequence as set forth in SEQ ID NO: 1 and referring to the numbering of SEQ ID NO: 1. Even more preferably, the polypeptide having phytase activity comprises at least eight or at least nine amino acid substitutions selected from the group consisting of 71L, 104R, 192A, 195F, 201W, 239K, 243Y, 337N, 360R and 365A compared to the amino acid sequence as set forth in SEQ ID NO: 1 and referring to the numbering of SEQ ID NO: 1. Most preferably, the polypeptide having phytase activity comprises the amino acid substitutions 71L, 104R, 192A, 195F, 201W, 239K, 243Y, 337N, 360R and 365A, compared to the amino acid sequence as set forth in SEQ ID NO: 1 and referring to the numbering of SEQ ID NO: 1.

In one embodiment, the polypeptide having phytase activity comprises at least one amino acid substitution selected from the group consisting of 192A, 195F, 243Y, 360R and 365A, compared to the amino acid sequence as set forth in SEQ ID NO: 1 and referring to the numbering of SEQ ID NO: 1.

In one embodiment, the polypeptide having phytase activity comprises the amino acid substitution 195F and optionally one or more additional amino acid substitutions selected from the group consisting of 71 L, 104R, 192A, 201W, 239K, 243Y, 337N, 360R and 365A, compared to the amino acid sequence as set forth in SEQ ID NO: 1 and referring to the numbering of SEQ ID NO: 1.

In one embodiment, the polypeptide having phytase activity comprises the amino acid substitution 365A and optionally one or more additional amino acid modifications selected from the group consisting of 71L, 104R, 192A, 195F, 201W, 239K, 243Y, 337N and 360R, compared to the amino acid sequence as set forth in SEQ ID NO: 1 and referring to the numbering of SEQ ID NO: 1.

In one embodiment, the polypeptide having phytase activity comprises the amino acid substitution 360R and optionally one or more additional amino acid substitutions selected from the group consisting of 71L, 104R, 192A, 195F, 201W, 239K, 243Y, 337N and 365A, compared to the amino acid sequence as set forth in SEQ ID NO: 1 and referring to the numbering of SEQ ID NO: 1.

In one embodiment, the polypeptide having phytase activity comprises the amino acid substitution 243Y and optionally one or more additional amino acid substitutions selected from the group consisting of 71L, 104R, 192A, 195F, 201W, 239K, 337N, 360R and 365A, compared to the amino acid sequence as set forth in SEQ ID NO: 1 and referring to the numbering of SEQ ID NO: 1.

In one embodiment, the polypeptide having phytase activity comprises the amino acid substitution at amino acid residue number 192A and optionally one or more additional amino acid substitutions selected from the group consisting of 71L, 104R, 195F, 201W, 239K, 243Y, 337N, 360R and 365A, compared to the amino acid sequence as set forth in SEQ ID NO: 1 and referring to the numbering of SEQ ID NO: 1.

In one embodiment, the polypeptide having phytase activity comprises at least one additional amino acid modification at an amino acid residue number selected from the group consisting of 30 and 45 compared to the amino acid sequence as set forth in SEQ ID NO: 1 and referring to the numbering of SEQ ID NO: 1.

Preferably, the polypeptide having phytase activity comprises additional amino acid modifications at the amino acid residues 30 and 45 compared to the amino acid sequence as set forth in SEQ ID NO: 1 and referring to the numbering of SEQ ID NO: 1.

In one embodiment, the polypeptide having phytase activity comprises amino acid modifications at the amino acid residues 30, 45 and 195 compared to the amino acid sequence as set forth in SEQ ID NO: 1 and referring to the numbering of SEQ ID NO: 1.

In one embodiment, the polypeptide having phytase activity comprises amino acid modifications at the amino acid residues 30, 45, 195 and 360 compared to the amino acid sequence as set forth in SEQ ID NO: 1 and referring to the numbering of SEQ ID NO: 1.

In one embodiment, the polypeptide having phytase activity comprises amino acid modifications at the amino acid residues 30, 45, 195 and 365 compared to the amino acid sequence as set forth in SEQ ID NO: 1 and referring to the numbering of SEQ ID NO: 1.

In one embodiment, the polypeptide having phytase activity comprises amino acid modifications at the amino acid residues 30, 45, 195 and 243 compared to the amino acid sequence as set forth in SEQ ID NO: 1 and referring to the numbering of SEQ ID NO: 1.

In one embodiment, the polypeptide having phytase activity comprises amino acid modifications at the amino acid residues 30, 45, 195, 360 and 365 compared to the amino acid sequence as set forth in SEQ ID NO: 1 and referring to the numbering of SEQ ID NO: 1.

In one embodiment, the polypeptide having phytase activity comprises amino acid modifications at the amino acid residues 30, 45, 195 and 365 compared to the amino acid sequence as set forth in SEQ ID NO: 1 and referring to the numbering of SEQ ID NO: 1.

In one embodiment, the polypeptide having phytase activity comprises amino acid modifications at the amino acid residues 30, 45, 192, 195, 360 and 365 compared to the amino acid sequence as set forth in SEQ ID NO: 1 and referring to the numbering of SEQ ID NO: 1.

In one embodiment, the polypeptide having phytase activity comprises at least one additional amino acid substitution at an amino acid residue number selected from the group consisting of 30 and 45 compared to the amino acid sequence as set forth in SEQ ID NO: 1 and referring to the numbering of SEQ ID NO: 1.

Preferably, the polypeptide having phytase activity comprises additional amino acid substitutions at the amino acid residues 30 and 45 compared to the amino acid sequence as set forth in SEQ ID NO: 1 and referring to the numbering of SEQ ID NO: 1.

In one embodiment, the polypeptide having phytase activity comprises amino acid substitutions at the amino acid residues 30, 45 and 195 compared to the amino acid sequence as set forth in SEQ ID NO: 1 and referring to the numbering of SEQ ID NO: 1.

In one embodiment, the polypeptide having phytase activity comprises amino acid substitutions at the amino acid residues 30, 45, 195 and 360 compared to the amino acid sequence as set forth in SEQ ID NO: 1 and referring to the numbering of SEQ ID NO: 1.

In one embodiment, the polypeptide having phytase activity comprises amino acid substitutions at the amino acid residues 30, 45, 195 and 365 compared to the amino acid sequence as set forth in SEQ ID NO: 1 and referring to the numbering of SEQ ID NO: 1.

In one embodiment, the polypeptide having phytase activity comprises amino acid substitutions at the amino acid residues 30, 45, 195 and 243 compared to the amino acid sequence as set forth in SEQ ID NO: 1 and referring to the numbering of SEQ ID NO: 1.

In one embodiment, the polypeptide having phytase activity comprises amino acid substitutions at the amino acid residues 30, 45, 195, 360 and 365 compared to the amino acid sequence as set forth in SEQ ID NO: 1 and referring to the numbering of SEQ ID NO: 1.

In one embodiment, the polypeptide having phytase activity comprises amino acid substitutions at the amino acid residues 30, 45, 195 and 365 compared to the amino acid sequence as set forth in SEQ ID NO: 1 and referring to the numbering of SEQ ID NO: 1.

In one embodiment, the polypeptide having phytase activity comprises amino acid substitutions at the amino acid residues 30, 45, 192, 195, 360 and 365 compared to the amino acid sequence as set forth in SEQ ID NO: 1 and referring to the numbering of SEQ ID NO: 1.

In one embodiment, the polypeptide having phytase activity comprises at least one additional amino acid substitution selected from the group consisting of 30L and 45P compared to the amino acid sequence as set forth in SEQ ID NO: 1 and referring to the numbering of SEQ ID NO: 1.

Preferably, the polypeptide having phytase activity comprises the additional amino acid substitutions 30L and 45P compared to the amino acid sequence as set forth in SEQ ID NO: 1 and referring to the numbering of SEQ ID NO: 1.

In one embodiment, the polypeptide having phytase activity comprises the amino acid substitutions 30L, 45P and 195F compared to the amino acid sequence as set forth in SEQ ID NO: 1 and referring to the numbering of SEQ ID NO: 1.

In one embodiment, the polypeptide having phytase activity comprises the amino acid substitutions 30L, 45P, 195F and 360R compared to the amino acid sequence as set forth in SEQ ID NO: 1 and referring to the numbering of SEQ ID NO: 1.

In one embodiment, the polypeptide having phytase activity comprises the amino acid substitutions 30L, 45P, 195F and 365A compared to the amino acid sequence as set forth in SEQ ID NO: 1 and referring to the numbering of SEQ ID NO: 1.

In one embodiment, the polypeptide having phytase activity comprises the amino acid substitutions 30L, 45P, 195F and 243Y compared to the amino acid sequence as set forth in SEQ ID NO: 1 and referring to the numbering of SEQ ID NO: 1.

In one embodiment, the polypeptide having phytase activity comprises the amino acid substitutions 30L, 45P, 195F, 360R and 365A compared to the amino acid sequence as set forth in SEQ ID NO: 1 and referring to the numbering of SEQ ID NO: 1.

In one embodiment, the polypeptide having phytase activity comprises the amino acid substitutions 30L, 45P, 195F and 365A compared to the amino acid sequence as set forth in SEQ ID NO: 1 and referring to the numbering of SEQ ID NO: 1.

In one embodiment, the polypeptide having phytase activity comprises the amino acid substitutions 30L, 45P, 192A, 195F, 360R and 365A compared to the amino acid sequence as set forth in SEQ ID NO: 1 and referring to the numbering of SEQ ID NO: 1.

Preferably, the polypeptide having phytase activity comprises the amino acid substitutions 30L, 45P, 71L, 104R, 192A, 195F, 201W, 239K, 243Y, 337N, 360R and 365A, compared to the amino acid sequence as set forth in SEQ ID NO: 1 and referring to the numbering of SEQ ID NO: 1.

In one embodiment, the polypeptide having phytase activity comprises the amino acid substitutions 30L, 45P and 195F and optionally one or more additional amino acid substitutions selected from the group consisting of 71 L, 104R, 192A, 201W, 239K, 243Y, 337N, 360R and 365A, compared to the amino acid sequence as set forth in SEQ ID NO: 1 and referring to the numbering of SEQ ID NO: 1.

In one embodiment, the polypeptide having phytase activity comprises the amino acid substitutions 30L, 45P and 365A and optionally one or more additional amino acid modifications selected from the group consisting of 71L, 104R, 192A, 195F, 201W, 239K, 243Y, 337N and 360R, compared to the amino acid sequence as set forth in SEQ ID NO: 1 and referring to the numbering of SEQ ID NO: 1.

In one embodiment, the polypeptide having phytase activity comprises the amino acid substitutions 30L, 45P and 360R and optionally one or more additional amino acid substitutions selected from the group consisting of 71L, 104R, 192A, 195F, 201W, 239K, 243Y, 337N and 365A, compared to the amino acid sequence as set forth in SEQ ID NO: 1 and referring to the numbering of SEQ ID NO: 1.

In one embodiment, the polypeptide having phytase activity comprises the amino acid substitutions 30L, 45P and 243Y and optionally one or more additional amino acid substitutions selected from the group consisting of 71L, 104R, 192A, 195F, 201W, 239K, 337N, 360R and 365A, compared to the amino acid sequence as set forth in SEQ ID NO: 1 and referring to the numbering of SEQ ID NO: 1.

In one embodiment, the polypeptide having phytase activity comprises the amino acid substitutions 30L, 45P and 192A and optionally one or more additional amino acid substitutions selected from the group consisting of 71L, 104R, 195F, 201W, 239K, 243Y, 337N, 360R and 365A, compared to the amino acid sequence as set forth in SEQ ID NO: 1 and referring to the numbering of SEQ ID NO: 1.

In one embodiment, the polypeptide having phytase activity comprises at least one additional amino acid modification at an amino acid residue number selected from the group consisting of 48, 111, 113, 209 and 366 compared to the amino acid sequence as set forth in SEQ ID NO: 1 and referring to the numbering of SEQ ID NO: 1.

In one embodiment, the polypeptide having phytase activity comprises additional amino acid modifications at the amino acid residues 48, 111, 113, 209 and 366 compared to the amino acid sequence as set forth in SEQ ID NO: 1 and referring to the numbering of SEQ ID NO: 1.

In one embodiment, the polypeptide having phytase activity comprises at least one additional amino acid substitution at an amino acid residue number selected from the group consisting of 48, 111, 113, 209 and 366 compared to the amino acid sequence as set forth in SEQ ID NO: 1 and referring to the numbering of SEQ ID NO: 1.

In one embodiment, the polypeptide having phytase activity comprises additional amino acid substitutions at the amino acid residues 48, 111, 113, 209 and 366 compared to the amino acid sequence as set forth in SEQ ID NO: 1 and referring to the numbering of SEQ ID NO: 1.

In one embodiment, the polypeptide having phytase activity comprises at least one additional amino acid substitution selected from the group consisting of 48E, 111G, 113K, 209S and T366E compared to the amino acid sequence as set forth in SEQ ID NO: 1 and referring to the numbering of SEQ ID NO: 1.

In one embodiment, the polypeptide having phytase activity comprises the additional amino acid substitutions 48E, 111G, 113K, 209S and 366E compared to the amino acid sequence as set forth in SEQ ID NO: 1 and referring to the numbering of SEQ ID NO: 1.

Preferably, the polypeptide having phytase activity comprises the amino acid substitutions 30L, 45P, 48E, 71 L, 104R, 111 G, 113K, 192A, 195F, 201W, 209S, 239K, 243Y, 337N, 360R, 365A and 366E, compared to the amino acid sequence as set forth in SEQ ID NO: 1 and referring to the numbering of SEQ ID NO: 1.

In one embodiment, the polypeptide having phytase activity comprises at least one additional amino acid modification at an amino acid residue number selected from the group consisting of 2, 76, 78, 89, 121, 138, 143, 152, 164, 200, 202, 207, 234, 242, 244, 247, 251, 256, 258, 261, 270, 314, 355 and 372 compared to the amino acid sequence as set forth in SEQ ID NO: 1 and referring to the numbering of SEQ ID NO: 1. In one embodiment, the polypeptide having phytase activity comprises at least two, at least three, at least four or at least five additional amino acid modifications at an amino acid residue number selected from the group consisting of 2, 76, 78, 89, 121, 138, 143, 152, 164, 200, 202, 207, 234, 242, 244, 247, 251, 256, 258, 261, 270, 314, 355 and 372 compared to the amino acid sequence as set forth in SEQ ID NO: 1 and referring to the numbering of SEQ ID NO: 1. In one embodiment, the polypeptide having phytase activity comprises at least six, at least seven, at least eight or at least nine additional amino acid modifications at an amino acid residue number selected from the group consisting of 2, 76, 78, 89, 121, 138, 143, 152, 164, 200, 202, 207, 234, 242, 244, 247, 251, 256, 258, 261, 270, 314, 355 and 372 compared to the amino acid sequence as set forth in SEQ ID NO: 1 and referring to the numbering of SEQ ID NO: 1. In one embodiment, the polypeptide having phytase activity comprises at least ten, at least eleven, at least 12 or at least 13 additional amino acid modifications at an amino acid residue number selected from the group consisting of 2, 76, 78, 89, 121, 138, 143, 152, 164, 200, 202, 207, 234, 242, 244, 247, 251, 256, 258, 261, 270, 314, 355 and 372 compared to the amino acid sequence as set forth in SEQ ID NO: 1 and referring to the numbering of SEQ ID NO: 1. In one embodiment, the polypeptide having phytase activity comprises at least 14, at least 15, at least 16 or at least 17 additional amino acid modifications at an amino acid residue number selected from the group consisting of 2, 76, 78, 89, 121, 138, 143, 152, 164, 200, 202, 207, 234, 242, 244, 247, 251, 256, 258, 261, 270, 314, 355 and 372 compared to the amino acid sequence as set forth in SEQ ID NO: 1 and referring to the numbering of SEQ ID NO: 1. In one embodiment, the polypeptide having phytase activity comprises at least 18, at least 19, at least 20, at least 21, at least 22 or at least 23 additional amino acid modifications at an amino acid residue number selected from the group consisting of 2, 76, 78, 89, 121, 138, 143, 152, 164, 200, 202, 207, 234, 242, 244, 247, 251, 256, 258, 261, 270, 314, 355 and 372 compared to the amino acid sequence as set forth in SEQ ID NO: 1 and referring to the numbering of SEQ ID NO: 1.

In one embodiment, the polypeptide having phytase activity comprises additional amino acid modifications at the amino acid residues 2, 76, 78, 89, 121, 138, 143, 152, 164, 200, 202, 207, 234, 242, 244, 247, 251, 256, 258, 261, 270, 314, 355 and 372 compared to the amino acid sequence as set forth in SEQ ID NO: 1 and referring to the numbering of SEQ ID NO: 1.

In one embodiment, the polypeptide having phytase activity comprises at least one additional amino acid substitution at an amino acid residue number selected from the group consisting of 2, 76, 78, 89, 121, 138, 143, 152, 164, 200, 202, 207, 234, 242, 244, 247, 251, 256, 258, 261, 270, 314, 355 and 372 compared to the amino acid sequence as set forth in SEQ ID NO: 1 and referring to the numbering of SEQ ID NO: 1. In one embodiment, the polypeptide having phytase activity comprises at least two, at least three, at least four or at least five additional amino acid substitutions at an amino acid residue number selected from the group consisting of 2, 76, 78, 89, 121, 138, 143, 152, 164, 200, 202, 207, 234, 242, 244, 247, 251, 256, 258, 261, 270, 314, 355 and 372 compared to the amino acid sequence as set forth in SEQ ID NO: 1 and referring to the numbering of SEQ ID NO: 1. In one embodiment, the polypeptide having phytase activity comprises at least six, at least seven, at least eight or at least nine additional amino acid substitutions at an amino acid residue number selected from the group consisting of 2, 76, 78, 89, 121, 138, 143, 152, 164, 200, 202, 207, 234, 242, 244, 247, 251, 256, 258, 261, 270, 314, 355 and 372 compared to the amino acid sequence as set forth in SEQ ID NO: 1 and referring to the numbering of SEQ ID NO: 1. In one embodiment, the polypeptide having phytase activity comprises at least ten, at least eleven, at least 12 or at least 13 additional amino acid substitutions at an amino acid residue number selected from the group consisting of 2, 76, 78, 89, 121, 138, 143, 152, 164, 200, 202, 207, 234, 242, 244, 247, 251, 256, 258, 261, 270, 314, 355 and 372 compared to the amino acid sequence as set forth in SEQ ID NO: 1 and referring to the numbering of SEQ ID NO: 1. In one embodiment, the polypeptide having phytase activity comprises at least 14, at least 15, at least 16 or at least 17 additional amino acid substitutions at an amino acid residue number selected from the group consisting of 2, 76, 78, 89, 121, 138, 143, 152, 164, 200, 202, 207, 234, 242, 244, 247, 251, 256, 258, 261, 270, 314, 355 and 372 compared to the amino acid sequence as set forth in SEQ ID NO: 1 and referring to the numbering of SEQ ID NO: 1. In one embodiment, the polypeptide having phytase activity comprises at least 18, at least 19, at least 20, at least 21, at least 22 or at least 23 additional amino acid substitutions at an amino acid residue number selected from the group consisting of 2, 76, 78, 89, 121, 138, 143, 152, 164, 200, 202, 207, 234, 242, 244, 247, 251, 256, 258, 261, 270, 314, 355 and 372 compared to the amino acid sequence as set forth in SEQ ID NO: 1 and referring to the numbering of SEQ ID NO: 1.

In one embodiment, the polypeptide having phytase activity comprises additional amino acid substitutions at the amino acid residues 2, 76, 78, 89, 121, 138, 143, 152, 164, 200, 202, 207, 234, 242, 244, 247, 251, 256, 258, 261, 270, 314, 355 and 372 compared to the amino acid sequence as set forth in SEQ ID NO: 1 and referring to the numbering of SEQ ID NO: 1.

In one embodiment, the polypeptide having phytase activity comprises at least one additional amino acid substitution selected from the group consisting of 2E, 76N, 78T, 89T, 121T, 138N, 143Y, 152G, 164E, 200N, 202S, 207E, 234V, 242N, 244S, 247K, 251N, 256H, 258N, 261H, 270Q, 314G, 355I and 372N compared to the amino acid sequence as set forth in SEQ ID NO: 1 and referring to the numbering of SEQ ID NO: 1. In one embodiment, the polypeptide having phytase activity comprises at least two, at least three, at least four or at least five additional amino acid substitutions at the amino acid residues selected from the group consisting of 2E, 76N, 78T, 89T, 121T, 138N, 143Y, 152G, 164E, 200N, 202S, 207E, 234V, 242N, 244S, 247K, 251N, 256H, 258N, 261H, 270Q, 314G, 355I and 372N compared to the amino acid sequence as set forth in SEQ ID NO: 1 and referring to the numbering of SEQ ID NO: 1. In one embodiment, the polypeptide having phytase activity comprises at least six, at least seven, at least eight or at least nine additional amino acid substitutions selected from the group consisting of 2E, 76N, 78T, 89T, 121T, 138N, 143Y, 152G, 164E, 200N, 202S, 207E, 234V, 242N, 244S, 247K, 251N, 256H, 258N, 261H, 270Q, 314G, 355I and 372N compared to the amino acid sequence as set forth in SEQ ID NO: 1 and referring to the numbering of SEQ ID NO: 1.

In one embodiment, the polypeptide having phytase activity comprises at least ten, at least eleven, at least 12 or at least 13 additional amino acid substitutions selected from the group consisting of 2E, 76N, 78T, 89T, 121T, 138N, 143Y, 152G, 164E, 200N, 202S, 207E, 234V, 242N, 244S, 247K, 251N, 256H, 258N, 261H, 270Q, 314G, 355I and 372N compared to the amino acid sequence as set forth in SEQ ID NO: 1 and referring to the numbering of SEQ ID NO: 1.

In one embodiment, the polypeptide having phytase activity comprises at least 14, at least 15, at least 16 or at least 17 additional amino acid substitutions selected from the group consisting of 2E, 76N, 78T, 89T, 121T, 138N, 143Y, 152G, 164E, 200N, 202S, 207E, 234V, 242N, 244S, 247K, 251N, 256H, 258N, 261H, 270Q, 314G, 355I and 372N compared to the amino acid sequence as set forth in SEQ ID NO: 1 and referring to the numbering of SEQ ID NO: 1. In one embodiment, the polypeptide having phytase activity comprises at least 18, at least 19, at least 20, at least 21, at least 22 or at least 23 additional amino acid substitutions selected from the group consisting of 2E, 76N, 78T, 89T, 121T, 138N, 143Y, 152G, 164E, 200N, 202S, 207E, 234V, 242N, 244S, 247K, 251N, 256H, 258N, 261H, 270Q, 314G, 355I and 372N compared to the amino acid sequence as set forth in SEQ ID NO: 1 and referring to the numbering of SEQ ID NO: 1.

In one embodiment, the polypeptide having phytase activity comprises the additional amino acid substitutions 2E, 76N, 78T, 89T, 121T, 138N, 143Y, 152G, 164E, 200N, 202S, 207E, 234V, 242N, 244S, 247K, 251N, 256H, 258N, 261H, 270Q, 314G, 355I and 372N compared to the amino acid sequence as set forth in SEQ ID NO: 1 and referring to the numbering of SEQ ID NO: 1.

In one embodiment, the polypeptide having phytase activity comprises the amino acid substitutions 2E, 71L, 76N, 78T, 89T, 104R, 121T, 138N, 143Y, 152G, 164E, 192A, 195F, 200N, 201W, 202S, 207E, 234V, 239K, 242N, 243Y, 244S, 247K, 251N, 256H, 258N, 261H, 270Q, 314G, 337N, 355I, 360R, 365A and 372N, compared to the amino acid sequence as set forth in SEQ ID NO: 1 and referring to the numbering of SEQ ID NO: 1.

In one embodiment, the polypeptide having phytase activity comprises the amino acid substitutions 2E, 30L, 45P, 71L, 76N, 78T, 89T, 104R, 121T, 138N, 143Y, 152G, 164E, 192A, 195F, 200N, 201W, 202S, 207E, 234V, 239K, 242N, 243Y, 244S, 247K, 251N, 256H, 258N, 261H, 270Q, 314G, 337N, 355I, 360R, 365A and 372N, compared to the amino acid sequence as set forth in SEQ ID NO: 1 and referring to the numbering of SEQ ID NO: 1.

In one embodiment, the polypeptide having phytase activity comprises the amino acid substitutions 2E, 30L, 45P, 48E, 71L, 76N, 78T, 89T, 104R, 111G, 113K, 121T, 138N, 143Y, 152G, 164E, 192A, 195F, 200N, 201W, 202S, 207E, 209S, 234V, 239K, 242N, 243Y, 244S, 247K, 251N, 256H, 258N, 261H, 270Q, 314G, 337N, 355I, 360R, 365A, 366E and 372N, compared to the amino acid sequence as set forth in SEQ ID NO: 1 and referring to the numbering of SEQ ID NO: 1.

The present invention also relates to a polypeptide having phytase activity or fragment thereof having phytase activity, wherein the polypeptide or fragment thereof has an amino acid sequence which is at least 95.9% identical, at least 96% identical, at least 96.1% identical, at least 96.2% identical, at least 96.3% identical, at least 96.4% identical, at least 96.5% identical, at least 96.6% identical, at least 96.7% identical, at least 96.8% identical, at least 96.9% identical, at least 97% identical, at least 97.1% identical, at least 97.2% identical, at least 97.3% identical, at least 97.4% identical, at least 97.5% identical, at least 97.6% identical, at least 97.7% identical, at least 97.8% identical, at least 97.9% identical, at least 98% identical, at least 98.1% identical, at least 98.2% identical, at least 98.3% identical, at least 98.4% identical, at least 98.5% identical, at least 98.6% identical, at least 98.7% identical, at least 98.8% identical, at least 98.9% identical, at least 99% identical, at least 99.1% identical, at least 99.2% identical, at least 99.3% identical, at least 99.4% identical, at least 99.5% identical, at least 99.6% identical, at least 99.7% identical, at least 99.8% identical, at least 99.9% identical or 100% identical to the full length amino acid sequence of the phytase according to SEQ ID NO: 2. In a preferred embodiment, the polypeptide having phytase activity has the amino acid sequence according to SEQ ID NO: 2. % identity is determined using the Needle program of the EMBOSS package (version 6.3.1.2 or later) with a gap open penalty of 10, a gap extension penalty of 0.5, and the EBLOSUM62 by the calculation: (Identical Residues×100)/(Length of Alignment−Total Number of Gaps in Alignment), which is also called “Longest_Identity” in programs output when parameter “-nobrief” is applied.

As discussed above, the phytase variants of the present invention show an increased thermostability. The thermostability can be described by the T50 value, i.e. the temperature at which the phytase retains 50% of its activity in comparison to the baseline activity. The T50 can be determined by incubating purified phytase samples at different temperatures such as 80° C. to 100° C. for a certain period such as 20 minutes, cooling the samples to 4° C. and determining phytase activity. The phytase activity values are then compared to the baseline activity, i.e. the activity at the lowest temperature tested. The determination of the T50 is also described in the examples herein. The phytase variants of the present invention have a T50 of at least 81.5° C., 82° C., 82.5° C. or 83° C. preferably of at least or 83.5° C., 84° C., 84.5° C., 85° C., 85.5° C., 86° C., 86.5° C., 87° C., 87.5° C., 88° C., 88.5° C., 89° C. or 89.5° C., more preferably of at least 90° C., 90.5° C., 91° C., 91.5° C., 92° C. or 92.5° C., most preferably of at least 93° C., 93.5° C., 94° C., 94.5° C., 95° C., 95.5° C., 96° C., 96.5° C. or 97° C. In one embodiment, the phytase variants of the present invention have a T50 of 81.5° C. to 97° C. or of 83.5° C. to 95° C. or of 86° C. to 93° C.

Further, the phytase variants of the present invention show a high stability at low pH. This is particularly advantageous, as the phytase activity does not decrease rapidly in the low pH environment of the stomach when the phytase is taken up with the animal feed. The stability of the phytase in the stomach can be simulated by incubating the phytase at a low pH of e.g. pH 1.2 in the presence of pepsin. The phytase variants of the present invention retain at least 50%, 55%, 60% or 65% of the phytase activity at TO when incubated with 3.8 mg/ml pepsin at pH 1.2 for 10 minutes. The phytase variants of the present invention retain at least 50%, 55% or 60% of the phytase activity at TO when incubated with 3.8 mg/ml pepsin at pH 1.2 for 20 minutes. Preferably, the phytase variants of the present invention retain at least 70%, 75% or 80% of the phytase activity at TO when incubated with 3.8 mg/ml pepsin at pH 1.2 for 10 minutes.

Additionally, the phytase variants of the present invention show a high activity against lower phytate esters. The activity against lower phytate esters can be determined by incubating the phytase with inositol phosphates with different numbers of phosphates, e.g. six (IP6), five (IP5), four (IP4), three (IP3) and two phosphates (IP2) and comparing the activity against inositol phosphates with five, four, three and/or two phosphates with the activity against inositol phosphates with six phosphates. The phytase variants of the present invention show an activity against inositol phosphate with five phosphate groups (IP5) of at least 70%, 72% or 74%, preferably 76% or 78% and most preferably at least 80% or 82% of the activity against inositol phosphate with six phosphate groups (IP6). The phytase variants of the present invention show an activity against inositol phosphate with four phosphate groups (IP4) of at least 30%, 32% 34%, 36% or 38%, preferably at least 40%, 42%, 44% or 46% and most preferably at least 48% or 50% of the activity against inositol phosphate with six phosphate groups (IP6). The phytase variants of the present invention show an activity against inositol phosphate with three phosphate groups (IP3) of at least 17%, 18% 19% or 20%, preferably at least 21%, 22%, 23% or 24% and most preferably at least 25% or 26% of the activity against inositol phosphate with six phosphate groups (IP6).

The present invention also relates to a polypeptide having phytase activity or a fragment thereof having phytase activity, wherein the polypeptide or fragment thereof having phytase activity has a melting temperature Tm of at least 101.5° C. as determined by differential scanning calorimetry using the conditions as described in Example 1.2.

In one embodiment, the polypeptide having phytase activity or a fragment thereof having phytase activity has a melting temperature Tm of at least 101.6° C., at least 101.7° C., at least 101.8° C., at least 101.9° C., at least 102.0° C., at least 102.1° C., at least 102.2° C., at least 102.3° C., at least 102.4° C., at least 102.5° C., at least 102.6° C., at least 102.7° C., at least 102.8° C., at least 102.9° C., at least 103.0° C., at least 103.1° C., at least 103.2° C., at least 103.3° C., at least 103.4° C., at least 103.5° C., at least 103.6° C., at least 103.7° C., at least 103.8° C., at least 103.9° C., at least 104.0° C., at least 104.1° C., at least 104.2° C., at least 104.3° C., at least 104.4° C., at least 104.5° C., at least 104.6° C., at least 104.7° C., at least 104.8° C., at least 104.9° C. or at least 105.0° C. as determined by differential scanning calorimetry using the conditions as described in Example 1.2.

In one embodiment, the polypeptide having phytase activity or a fragment thereof having phytase activity has a melting temperature Tm of at least 101.5° C. to 105.0° C., at least 102.0° C. to 105.0° C., at least 102.5° C. to 105.0° C., at least 103.0° C. to 105.0° C., at least 103.5° C. to 105.0° C., at least 104.0° C. to 105.0° C. or at least at least 104.5° C. to 105.0° C. as determined by differential scanning calorimetry using the conditions as described in Example 1.2.

The present invention also relates to an isolated nucleic acid molecule comprising a nucleic acid sequence encoding the phytase of the present invention. The skilled person knows how to construct a nucleic acid molecule when the amino acid sequence of a protein is known. In particular, the construction of the nucleic acid molecule involves back-translating the amino acid sequence of the protein into a nucleic acid sequence using the three-letter genetic code and optionally taking into account the codon usage of the host cell in which the protein is to be expressed using the nucleic acid molecule.

The term “isolated nucleic acid molecule” refers to a nucleic acid molecule that is identified and separated from at least one contaminant nucleic acid molecule with which it is ordinarily associated in the environment in which it was produced. Preferably, the isolated nucleic acid is free of association with all components associated with the production environment.

In one embodiment, the nucleic acid sequence encoding the phytase variant of the present invention is codon-optimized. In codon-optimized nucleic acid sequences the codons are selected to reflect the typical codon usage in the host cell into which the nucleic acid molecule comprising said nucleic acid sequence is introduced without altering the sequence of the polypeptide which is encoded by the nucleic acid sequence. In one embodiment, the nucleic acid sequence encoding the phytase variant of the present invention is codon-optimized for expression in E. coli. In another embodiment, the nucleic acid sequence encoding the phytase variant of the present invention is codon-optimized for expression in the Thermothelomyces thermophilus C1 strain. In yet another embodiment, the nucleic acid sequence encoding the phytase variant of the present invention is codon-optimized for expression in Aspergillus niger.

The present invention also relates to a vector comprising the isolated nucleic acid molecule of the present invention.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

Expression vectors contain elements for the expression of the nucleic acids such as suitable promoters. The promoters are operably linked to the nucleic acid sequence which is to be expressed, i.e. the nucleic acid sequence encoding the phytase variant of the present invention. The term “operably linked” refers to the association of nucleic acid sequences on a single nucleic acid molecule so that the function of one is affected by the other. For example, a promoter is operably linked with a coding sequence when it is capable of affecting the expression of that nucleic acid sequence, i.e., the coding sequence is under the transcriptional control of the promoter. In addition, the expression vectors typically contain a selection marker gene under the transcriptional control of suitable promoter to enable the distinction of cells which contain the expression vector from cells which do not contain the expression vector. The elements and methods needed to construct expression vectors which are suitable for expressing a recombinant protein such as the phytase of the present invention are well-known to the skilled person.

The expression vector is used to transform, i.e. genetically modify, suitable host cells. The skilled person is aware of methods for introducing the expression vectors into the host cells. After transformation the cells are subjected to selection by treatment with a suitable agent based on the selection marker(s) encoded by the expression vector(s) to identify the stably transformed cells which contain the expression vector.

The present invention also relates to a host cell comprising the isolated nucleic acid molecule or the vector of the present invention.

The terms “host cell,” “host cell line,” and “host cell culture” are used interchangeably and refer to cells into which an exogenous nucleic acid or an expression vector has been introduced, including the progeny of such cells. Host cells include “transformants” and “transformed cells,” which include the primary transformed cell and progeny derived therefrom without regard to the number of passages. Suitable host cells for expressing the phytase of the present invention include, but are not limited to, cells from the genus Thermothelomyces, Aspergillus, Pichia, Trichoderma, Hansenula, Saccharomyces, Bacillus, Escherichia, Kluyveromyces, and Schizosaccharomyces. In one embodiment, the phytase of the present invention is expressed in Escherichia coli. In one embodiment, the phytase of the present invention is expressed in Escherichia coli and is not glycosylated. In one embodiment, the phytase of the present invention is expressed in the Thermothelomyces thermophilus C1 strain. In one embodiment, the phytase of the present invention is expressed in the Thermothelomyces thermophilus C1 strain and is glycosylated. In one embodiment, the phytase of the present invention is expressed in Aspergillus niger In one embodiment, the phytase of the present invention is expressed in Aspergillus niger and is glycosylated.

The present invention also relates to a method for producing the polypeptide or fragment thereof having phytase activity of the present invention, comprising culturing the host cell of the present invention under suitable conditions, optionally further comprising recovering the polypeptide.

After fermentation of the host cell, the fermentation broth is obtained and the host cells and solids are removed by conventional separation techniques such as filtration, centrifugation, microfiltration, rotary vacuum drum filtration, ultrafiltration, centrifugation followed by ultrafiltration, extraction, or chromatography, thus yielding a phytase solution.

The phytase may then be recovered by methods such as affinity chromatography, ion-exchange chromatography, hydrophobic interaction chromatography, two-phase partitioning, ethanol precipitation, reverse phase H PLC, chromatography on silica or on a cation-exchange resin such as DEAE, chromatofocusing, SDS-PAGE, ammonium sulfate precipitation, and gel filtration.

The present invention also relates to an animal feed additive or animal feed comprising at least one phytase of the present invention.

A “feed” as used herein refers to any natural or artificial diet, meal or the like or components of such meals intended or suitable for being eaten, taken in, digested, by a non-human animal, respectively. Preferably, the term “feed” is used with reference to products that are fed to animals in the rearing of livestock. The terms “feed” and “animal feed” are used interchangeably herein. In one embodiment, animals which are fed with the feed are non-ruminant, i.e., monogastric animals including, but not limited to, pigs and swine, such as piglets, growing pigs, sows; poultry such as turkeys, ducks, chicken, broiler chicks, layers; fish such as salmon, trout, tilapia, catfish and carps; and crustaceans such as shrimps and prawns. In another embodiment, animals which are fed with the feed are ruminant animals including, but not limited to, cattle, young calves, goats, sheep, giraffes, bison, moose, elk, yaks, water buffalo, deer, camels, alpacas, llamas, antelope, pronghorn and nilgai.

A “feed additive” as used herein refers to one or more ingredients, products of substances (e.g., cells), used alone or together, in nutrition, e.g., to improve the quality of a food (e.g., an animal feed), to improve an animal's performance and/or health, and/or to enhance digestibility of a food or materials within a food.

In one aspect, the feed additive may be in the form of a granulated particle. Such granulated particles may be produced by a process selected from the group consisting of high shear granulation, drum granulation, extrusion, spheronization, fluidized bed agglomeration, fluidized bed spray coating, spray drying, freeze drying, prilling, spray chilling, spinning disk atomization, coacervation, tableting, or any combination of the above processes. The particles may have a mean diameter of 50 microns to 2000 microns.

In one aspect, the animal feed additive or animal feed further comprises at least one additional enzyme which may be selected from the group consisting of cellulase, xylanase, glucanase, mannanase, protease, amylase and a second phytase, or other feed ingredients such as at least one vitamin, such as Vitamin A or E.

Xylanase refers to a class of enzymes that degrade the linear polysaccharide β-1,4-xylan into xylose, thus breaking down hemicellulose, one of the major components of plant cell walls. In one embodiment, the xylanase may be any commercially available xylanase. The xylanase may be an endo-1,4-β-d-xylanase (classified as E. C, 3.2.1.8) or a 1,4β-xylosidase (classified as E.CG. 3.2.1.37). AII E. C. enzyme classifications referred to herein relate to the classifications provided in Enzyme Nomenclature—Recommendations (1992) of the nomenclature committee of the International Union of Biochemistry' and Molecular Biology—ISBN 0-12-226164-3.

In one embodiment, the xylanase may be a xylanase from Bacillus, Trichoderma, Therinomyces, Aspergillus, Humicola and Penicillium. In one embodiment, the xylanase may be a mixture of two or more xylanases. In still another embodiment, the xylanase is an endo-1,4-β-xylanase or a 1,4-β-xylosidase. In one embodiment, the xylanase is a xylanase according to EP 1319079 B1 or a variant thereof with an amino acid sequence having at least 80% sequence identity thereto or a fragment thereof and having xylanase activity. In one embodiment, the xylanase is the xylanase according to SEQ ID NO: 3 or a variant thereof with an amino acid sequence having at least 80% sequence identity thereto or a fragment thereof and having xylanase activity.

Mannanase refers to a class of enzymes that degrade mannans, i.e. polysaccharides made from mannose which is a simple sugar which are present widely in plant tissues. In one embodiment, the mannanase may be any commercially available mannanase. In one embodiment, the mannanase is a β-mannanase (EC 3.2.1.22). In one embodiment, the mannanase is an endo-1,4-β-D-mannanase. In one embodiment, the mannanase is a mannanase according to EP 2857490 B1 or EP 2052078 B1 or a variant thereof with an amino acid sequence having at least 80% sequence identity thereto and having mannanase activity. In one embodiment, the mannanase is the mannanase according to SEQ ID NO: 4 or a variant thereof with an amino acid sequence having at least 80% sequence identity thereto or a fragment thereof and having mannanase activity.

Glucanase refers to a class of enzymes that break down large polysaccharides by hydrolysis, resulting in linear polysaccharides made up of up to 1200 glucose monomers linked by glycosidic bonds. In one embodiment, the glucanase may be any commercially available glucanase. In one embodiment, the glucanase is an endo-1,4-β-glucanase. In one embodiment, the glucanase is the glucanase according to EP1272643 B2 or EP1621628 B1 or a variant thereof with an amino acid sequence having at least 80% sequence identity thereto and having glucanase activity. In one embodiment, the glucanase is the glucanase according to SEQ ID NO: 5 or a variant thereof with an amino acid sequence having at least 80% sequence identity thereto or a fragment thereof and having glucanase activity.

Cellulase refers to a class of enzymes which degrade cellulose by hydrolysing the 1,4-beta-D-glycosidic linkages, resulting in monosaccharides, shorter polysaccharides and oligosaccharides. In one embodiment, the cellulase may be any commercially available cellulase. In one embodiment, the cellulase may be an endocellulase (EC3.2.1.4) which randomly cleaves internal bonds within the cellulose molecule. In one embodiment, the cellulase may be an exocellulase (EC 3.2.1.91) which cleaves units from the ends of the cellulose molecule.

The “second phytase” is a phytase which has a different amino acid sequence as the phytase of the present invention, which is able to cleave phytate esters.

The present invention also relates to the use of the phytase of the present invention or the animal feed additive of the present invention in an animal feed.

The present invention also relates to the use of the phytase of the present invention or the animal feed additive or animal feed of the present invention reducing the phosphate content in the slurry of livestock or for improving animal performance.

The embodiments described are intended to illustrate and to give a better understanding of the invention and are in no way to be construed as limiting. Further features of the invention result from the description hereinbelow of preferred embodiments in conjunction with the dependent claims. In this context, the individual features of the invention may, in one embodiment, be realized in each case individually or together and are no limitation whatsoever of the invention to the described embodiment. The wording of the patent claims is hereby expressly made subject matter of the description.

### EXAMPLES

### 1. Methods

### 1.1. Determination of Phytase Activity

The phytase activity was determined in microtiter plates. The enzyme sample with a concentration of 1 mg/ml was diluted 100-fold in reaction buffer (100 mM Na-acetate, pH 5.5). 50 μL of the enzyme solution were incubated with 950 μL substrate solution (4 mM sodium phytate (Sigma P3168) in reaction buffer) at 37° C. 50 μL aliquots of the reaction mixture were withdrawn at time intervals over 8 minutes and immediately quenched in a microtiter plate containing 50 μL of Stop/Color Reagent (5 mM ammonium vanadate, 20.25 mM ammonium molybdate, nitric acid). The color was allowed to develop for at least 15 minutes before the OD₄₁₅ₙₘ is read. One unit is defined by the moles of phosphate released per minute by the enzyme, under the conditions of the assay. The amount of liberated phosphate was determined via a calibration curve of the color reaction with a phosphate solution of known concentration.

### 1.2 Determination of the Melting Point of Phytases by Differential Scanning Calorimetry

The melting point of phytase variants was determined by differential scanning calorimetry using a CSC6100 Nano-Differential Scanning Calorimeter II (N-DSC) (Calorimetry Science Corporation, Lindon, Utah, USA). 1 mL of purified phytase in 50 mM Na-Acetate buffer (pH 5.5) and buffer control (50 mM Na-Acetate) were degassed under vacuum for 15 minutes to remove dissolved gas. About 600 μL of purified sample were loaded into the “sample cell” and 600 μL of buffer were loaded into the “reference cell.” The cells were pressurized to 3.0 ATMs and allowed to equilibrate prior to initiating a scan from 25° C.-115° C. at a rate of 1° C./min up and down. Melting temperature (Tₘ) was determined using the Nano-DSC Model 6100/6300 Data Acquisition and Analysis Software (Version 2.6.0.17) assessing the temperature inflection point in μW measurement during the scan.

### 1.3 Determination of T50

Purified phytase samples (100-fold dilution of a 1 mg/ml solution) in 50 mM Na-acetate buffer were challenged at different temperatures within the range of 80-100° C. or 75-100° C. for 20 minutes, then cooled to 4° C. Samples were then measured for phytase activity using the standard microtiter assay described in 1.1 above and values were compared to the baseline 80° C. results. T₅₀ thermal stability is the temperature at which the enzyme has 50% residual activity.

### 1.4 Determination of Activity on Different Phytate Esters

The phytase activity was determined in microtiter plates. The enzyme sample with a concentration of 1 mg/ml was diluted 100-fold in reaction buffer (50 mM Na acetate, pH 5.5). 50 μL of the enzyme solution were incubated with 950 μL of purified substrate (4 mM IP6, 4 mM IP5, 4 mM IP4 or 4 mM IP3, respectively, in reaction buffer) at 37° C. 50 μL aliquots of the reaction mixture were withdrawn at time intervals over 10 minutes and immediately quenched in a microtiter plate containing 50 μL of Stop/Color Reagent (5 mM ammonium vanadate, 20.25 mM ammonium molybdate, nitric acid). The color was allowed to develop for at least 15 minutes before the OD₄₁₅ₙₘ is read. One unit is defined by the moles of phosphate released per minute by the enzyme, under the conditions of the assay. The amount of liberated phosphate was determined via a calibration curve of the color reaction with a phosphate solution of known concentration.

### 1.5 Determination of Activity at Low pH in the Presence of Pepsin

A simulated gastric fluid (SGF) was prepared by preparing a 222 mg/ml NaCl solution and titrating it to pH 1.2 with HCl. 3.8 mg pepsin (P6887) was mixed with 5 ml of SGF to obtain a dose of 10 pepsin activity units/μg of test protein. 665 μl of this mixture were dispensed into a test tube and incubated at 37° C. for 3 minutes. The in vitro digestibility reaction was initiated by adding 35 μl of test protein to the pre-heated mixture. The reaction was terminated by removing 100 μl of the reaction sample and mixing it with 35 μl of 200 mM NaHCO₃ (pH 11.0) at different time-points, i.e. after 1, 3, 5, 10, 15 and 30 minutes. The untreated control sample was pre-quenched by raising the pH to inactivate the pepsin by mixing 35 μl of 200 mM NaHCO₃ with 95 μl SGF with pepsin and adding 5 μl of test sample. The enzyme activity was determined using terminated reaction material which was compared to the untreated enzyme control sample.

### 1.6 Isolation of Phytases

Secreted phytases were dialyzed in 50 mM TRIS (pH 8.5), 50 mM KCl buffer and filter sterilized. Using an AKTA HPLC Purifier System, the phytase sample was loaded and trapped onto a 5 mL HiTrap Q HP (Cytiva) column. The phytase protein was then eluted off the column using a salt gradient from 50 mM to 600 mM KCl in 1 mL fractions. The fractions with highest purity and concentration of eluted phytase were then collected and dialyzed in 50 mM Na-Acetate pH 5.5. The protein concentration was determined based on 260/280 nm absorbance and corrected for the molar extinction coefficient associated to specific amino acid protein sequence.

### 2. Generation and Analysis of Single Mutants

Single mutants were generated by gene site saturation mutagenesis of Seq ID NO: 1 prior art phytase and expressed in E. coli strain HMS174(DE3)pLysS using the expression vector pET22b. The phytase mutants were isolated as described in section 1.6 and analyzed for their thermostability as described in section 1.3. The thermostability was compared to the thermostability of the prior art thermostable phytase according to SEQ ID NO: 1 as disclosed in WO 2006/043178.

### 3. Fermentative Production of the Phytase in an E.Coli Host Cell

Phytase production in E. coli is achieved by using HMS174(DE3)pLys competent cells (Novagen) in combination with the pET-22b vector system that provides multiple mechanisms of expression regulation. Phytase gene is cloned downstream of the lacUV5 promoter which is recognized by the bacteriophage T7 RNA polymerase. Expression of phytase is induced by the addition of IPTG, which mimics lactose and binds the Lacl repressor.

Competent cells are transformed by mixing DNA plasmid (10-50 ng/μL DNA) with cells and heat shocking the transformation mixture at 42° C. for 30 minutes, followed by an immediate transfer back on ice for a minimum of 2 minutes. 100 μL of SOC medium (Teknova) are added to the transformation mixture and incubated for 90 minutes at 37° C. in a rotating incubator. 250 μl of the transformed mixture are plated on a LB Carb100Cm134 agar plate and spread evenly with glass beads. The agar plates are incubated for about 20 hours at 37° C. An isolated transformed colony is picked from the agar plate and used to inoculate 5 mL LB Carb100Cm134, folowed by incubation of the cell culture overnight at 37° C. in a rotating incubator. 100 mL LB Carb100Cm134 are seeded with overnight culture at a target cell optical density of 0.05 OD600. The culture is allowed to grow until 0.5 OD600 and then induced with 1 mM IPTG. Afterwards the culture is allowed to grow overnight at 37° C. in a rotating incubator. The cell culture is pelleted by centrifugation and the supernatant is discarded. The cell pellet is kept on ice and then the cells are lysed to release enzyme by adding B-Per cell lysis buffer (Thermo Scientific)) for one hour. The cell lysate is centrifuged and clear lysate is dialyzed in 100 mM TRIS pH 8.2 buffer. The dialyzed enzyme sample is purified using standard protein purification techniques such as FPLC (AKTA) coupled with Hi Trap Q column (Cytiva Life Sciences) and the bound enzyme is eluted with a 1 M NaCl gradient. Purified enzyme fractions are pooled and dialyzed overnight in 100 mM Na-Acetate pH 5.5. The dialyzed enzyme sample is used in the enzyme assays described herein.

### 4. Fermentative Production of the Phytase in a Fungal Host Cell

To express the phytase variant of the present invention in Aspergillus niger; an expression construct is first prepared which comprises the phytase gene under the control of the A. niger glucoamylase (glaA) promoter, flanked by the noncoding 3′-glaA region. In this manner, the construct is intended for integration into the 3′-glaA region in A. niger. The signal sequence used for the extracellular protein secretion is the signal sequence of the A. ficuum phytase. The cloning of the expression construct is based on the plasmid pGBGLA-53 (also referred to as pGBTOPFYT-1 in WO 98/46772), which is described in detail in EP 0 635 574 B1. With the aid of PCR-based cloning techniques known to a person skilled in the art, the gene segment of the A. ficuum phytase, which codes for the mature phytase protein starting with the amino acid sequence ASRNQSS, in pGBGLA-53 is replaced by a synthetic gene, which codes for the mature phytase variant of the present invention and is adapted to the codon usage of Aspergillus niger The cotransformation of the linear expression cassette, isolated from the resulting plasmid using HindIII, together with an amdS marker cassette, isolated from the plasmid pGBLA50 (EP 0 635 574 B1)/pGBAAS-1 (name of the same plasmid in WO 98/46772), into a glaA-deleted A. niger expression strain and the subsequent expression of the phytase in shake flasks is performed as described in the two cited patent specifications. The phytase activity in the culture supernatant is determined daily after the cells have been centrifuged off. The maximum activity is obtained between day 3 and day 6.

1. A polypeptide having phytase activity or fragment thereof having phytase activity, wherein the polypeptide or fragment thereof has an amino acid sequence which is at least 94% identical to the amino acid sequence as set forth in SEQ ID NO: 1 and wherein the polypeptide comprises at least one amino acid modification at an amino acid residue number selected from the group consisting of 71, 104, 192, 195, 201, 239, 243, 337, 360 and 365, compared to the amino acid sequence as set forth in SEQ ID NO: 1 and referring to the numbering of SEQ ID NO: 1.

2. The polypeptide or fragment thereof of claim 1, wherein the at least one amino acid modification is an amino acid substitution selected from the group consisting of 71 L, 104R, 192A, 195F, 201W, 239K, 243Y, 337N, 360R and 365A.

3. The polypeptide or fragment thereof of claim 1, having at least one additional amino acid modification at an amino acid residue number selected from the group consisting of 30, 45, 48, 111, 113, 209 and 366 compared to the amino acid sequence as set forth in SEQ ID NO: 1 and referring to the numbering of SEQ ID NO: 1.

4. The polypeptide or fragment thereof of claim 3, wherein the at least one additional amino acid modification is an amino acid substitution selected from the group consisting of 30L, 45P, 48E, 111G, 113K, 209S and 366E.

5. The polypeptide or fragment thereof of claim 1, having at least one additional amino acid modification at an amino acid residue number selected from the group consisting of 2, 76, 78, 89, 121, 138, 143, 152, 164, 200, 202, 207, 234, 242, 244, 247, 251,256,258,261, 270, 314, 355 and 372 compared to the amino acid sequence as set forth in SEQ ID NO: 1 and referring to the numbering of SEQ ID NO: 1.

6. The polypeptide or fragment thereof of claim 5, wherein the at least one additional amino acid modification is an amino acid substitution selected from the group consisting of 2E, 76N, 78T, 89T, 121T, 138N, 143Y, 152G, 164E, 200N, 202S, 207E, 234V, 242N, 244S, 247K, 251 N, 256H, 258N, 261H, 270Q, 314G, 355I and 372N.

7. A polypeptide having phytase activity or fragment thereof, wherein the polypeptide or fragment thereof has a melting temperature Tm of at least 101.5° C. as determined by differential scanning calorimetry using the conditions as described in Example 1.2.

8. A polypeptide having phytase activity or fragment thereof having phytase activity, wherein the polypeptide or fragment thereof has an amino acid sequence which is at least 95.9% identical to the amino acid sequence as set forth in SEQ ID NO: 2.

9. An isolated nucleic acid molecule comprising a nucleic acid sequence encoding the polypeptide of claim 1.

10. An expression vector comprising the nucleic acid molecule of claim 9.

11. A host cell comprising the nucleic acid molecule of claim 9.

12. A method for producing a polypeptide, comprising culturing a host cell under suitable conditions, optionally further comprising recovering the polypeptide,
wherein the host cell comprises a nucleic acid molecule comprising a nucleic acid sequence encoding a polypeptide having phytase activity, wherein the polypeptide has an amino acid sequence which is at least 94% identical to the amino acid sequence as set forth in SEQ ID NO: 1 and wherein the polypeptide comprises at least one amino acid modification at an amino acid residue number selected from the group consisting of 71, 104, 192, 195, 201, 239, 243, 337, 360 and 365, compared to the amino acid sequence as set forth in SEQ ID NO: 1 and referring to the numbering of SEQ ID NO: 1.

13. An animal feed additive or animal feed comprising at least one phytase according to claim 1, optionally further comprising at least one additional enzyme, further optionally wherein the at least one additional enzyme is selected from the group consisting of cellulase, xylanase, glucanase, mannanase and a second phytase.

14. (canceled)

15. (canceled)
